(19) United States
(12) Patent Application Publication
(10) Pub. No.: US 2015/0250656 A1
(43) Pub. Date: Sep. 10, 2015

(54) SANITARY BAG

(71) Applicant: McAirlaid's Vliesstoffe GmbH, Berlingerode (DE)

(72) Inventor: Alexander Maksimow, Steinfurt (DE)

(21) Appl. No.: 14/432,348

(22) PCT Filed: Sep. 27, 2013
(86) PCT No.: PCT/EP2013/070241

§ 371 Date: Mar. 30, 2015

(30) Foreign Application Priority Data
Oct. 1, 2012 (DE) 20 2012 103 745.9
Apr. 3, 2013 (DE) 20 2013 101 413.3

(51) Int. Cl. A61F 13/47; A61F 13/471

(52) U.S. Cl. CPC A61F 13/4704; A61F 13/471; A61F 2013/4518

(57) ABSTRACT

A sanitary bag for receiving at least one part of the body, in particular the penis, is claimed, which sanitary bag is composed at least of a blank (2) with two portions (A, B) which each have the shape of a quadrilateral and are folded along a fold line (F1, F2), wherein the two portions (A and B) have the same length from the fold line (F1, F2) to their edge (4, 5, 6, 7) facing away from the fold line, characterized in that an absorbent insert (3) is arranged on the inside of the blank (2).

The sanitary bag has the advantage that it is composed of simple geometric shapes and permits simple and cost-effective production, also with high-speed machines.

The present invention relates to a sanitary bag for receiving at least one part of the body, in particular the penis.

Many products for collecting bodily fluids, e.g. urine, are known on the market. Particular mention may be made of liners, diapers for babies and infants, and incontinence diapers. A common aspect of these products is that they are generally available on the market as so-called unisex products, the basic shape being adapted to the human anatomy. There are also many products specially developed for women, whereas aids developed for men are virtually unknown or are designed in such a way that they do not provide wearing comfort.

DE 698 09 666, for example, discloses a urine-receiving pad for men, which is formed by a bag provided with an opening and consists of at least one laminated sheet comprising a liquid-permeable inner layer, a liquid-impermeable outer layer, and an absorbent core provided between the two layers. This bag can be applied to a penis by inserting the penis into the opening, wherein flat or linearly elastic elements are arranged in pairs along an opening edge, such that the elastic elements, in a state in which they are deformed by buckling, engage the penis by means of elastic restoring forces.

DE 10 2008 020 606 B1 discloses a sanitary article of the type mentioned at the outset, which sanitary article has a sleeve-shaped main body for attaching to the body part, wherein the main body has an opening larger than the body part, and this main body has a steeply rising flank which, after reaching a peak, merges into a flat trailing edge, and the main body has a flap, wherein the opening can be made smaller by turning the flap back over the area of the main body located over the body part. The main body is usually cone-shaped.

A common aspect of the products known from the prior art is that they have a shape that makes industrial production complicated and therefore expensive.

The object of the present invention was therefore to make available a sanitary bag of the type mentioned at the outset, which is composed of simple geometric shapes and permits simple and cost-effective production, also with high-speed machines.

The present invention therefore relates to a sanitary bag for receiving at least one part of the body, in particular the penis, which sanitary bag is composed at least of a blank with two portions A and B which each have the shape of a quadrilateral and are folded along a fold line, wherein the two portions have the same length from the fold line to their edge facing away from the fold line, characterized in that an absorbent insert is arranged on the inside of the blank.

The sanitary bag according to the invention is composed of a blank which is folded along a fold line, wherein this folded shape forms the outer shape of the sanitary bag. The blank will usually be obtained from web material. To permit production in high-speed machines and also to minimize the amount of scrap, i.e. the non-usable remainders of the material from which the blank is cut out, the blank has the simplest possible geometric shape. Preferably, the portions each have the shape of a quadrilateral.

In a preferred embodiment, the blank has the shape of a square or a rectangle. This shape has the advantage of causing less scrap material compared to other shapes.

The blank is folded along the fold line, such that the portions are superposed. The edges of the blank that lie opposite the fold line form the opening for receiving the body part. The portions preferably have the same side length from the fold line as far as their edge facing away from the fold line.

If the blank is present in the shape of a square or rectangle, i.e. with four right angles, the fold line can either be located at the bottom of the sanitary bag, i.e. the edges of the blank that lie opposite the fold line form the opening for receiving the body part, or the fold line is located at a right angle to the opening. If the blank is present in the shape of a rectangle or a square, the blank can be folded along one of the two fold lines. If the blank is present in the form of a quadrilateral, the portions each having the shape of a trapezoid, the fold line forms the bottom portion of the article according to the invention.

The superposed edges of the portions adjoining the fold line are connected to each other. This connection can be made differently depending on the material from which the blank is made. For example, the edges can be connected to each other by gluing or welding, and in some cases also by sewing. This connection of the edges is preferably watertight, such that an escape of the bodily fluid through this connection of the edges is substantially avoided, preferably completely avoided.

In a preferred embodiment, the connecting edge is at a distance from the opening edge of the opening, i.e. the connection ends before the opening edge. In this way, a portion remains unclosed from the weld seam to the opening, as a result of which the opening is widened. This widening facilitates the insertion of the penis into the opening, i.e. the handling of the sanitary bag according to the invention.

The material from which the blank is made should be impermeable to liquids, the aim being to prevent urine or other bodily fluid from passing through the sanitary bag according to the invention in the direction of the clothes and soiling the latter. For optimal wearing comfort, it is preferable if the blank is made of a breathable material, i.e. a material that is permeable to water vapor but not to liquids. The material for the blank can be, for example, a breathable film, such as a perforated two-dimensional and/or three-dimensional film, a breathable SMS, nonwovens of natural or synthetic fibers, and/or a laminate of different materials, for example of nonwoven and breathable film (BIBS films=breathable film textile backsheet), as are also known for the production of the outsides of customary incontinence products.

According to the invention, an absorbent insert is arranged on the inside of the blank. The absorbent insert extends at least over a part of one of the portions; the absorbent insert preferably extends over both portions. However, the absorbent insert is preferably offset inward with respect to the edges. A single absorbent insert or several absorbent inserts can be arranged on the blank. In a preferred configuration, a single absorbent insert extends over both portions A and b, its edge being offset inward with respect to the edges. With respect to the edges that form the superposed closing edge, the distance between the absorbent insert and the edge should be so great that the superposed edges can be connected to each other without inclusion of the absorbent insert. The distance of the absorbent insert from the opening edge can be slightly greater, and this distance forms the bearing surface for the body part and should be chosen such that good wearing comfort is ensured.

In one possible embodiment, the sanitary bag according to the invention is multilayered and, in addition to the first blank, which usually faces outward, and to the absorbent insert on the inside, i.e. in the direction of the body part, it also has a further blank arranged over the absorbent insert. This blank is preferably made of a material permeable to liquids. The bodily fluid passes through this further blank in the direction of the absorbent insert. The purpose of this further layer is to quickly transport the bodily fluid away from the body part in the direction of the absorbent insert, where the fluid is taken up and stored. The first blank and the further blank preferably have the same size. The absorbent insert is encased by the two blanks. The two blanks can be connected to each other via a suitable connection, for example by welding with ultrasound. It is not necessary that the blanks are connected to each other in a watertight manner. The blanks should be larger than the absorbent insert, such that the absorbent insert is not included in the connection of the edges of the blanks.

The material from which the further blank is made is preferably a material permeable to liquids, for example a nonwoven, preferably a polypropylene/polyethylene nonwoven that is stretchable. The stretchability has the advantage that, when the absorbent body takes up liquid and its volume increases, it also does not change the surface of this further blank and gives the absorbent body room, without this further blank or the outwardly directed blank tearing.

For this reason, it has also proven advantageous if the absorbent core is slightly smaller than the volume formed by the outer blank and the further blank.

This absorbent insert serves to take up and permanently absorb the bodily fluid. Rewetting in the direction of the body part should preferably be avoided as far as possible. This absorbent insert is therefore preferably made from a material which is able not only to absorb bodily fluids, such as urine, but also to store them. Materials that are very suitable are those composed of oriented and/or unoriented fibers, in particular nonwovens of pulp and pulp-related materials, and also synthetic absorbent materials. The absorbent insert is preferably a nonwoven of cellulose fibers, such as an airlaid.

The absorbency of the absorbent core can be increased if it contains particles of superabsorbent polymers. For example, these can be incorporated directly during the production of the absorbent core.

In one possible embodiment, the absorbent insert is produced from a fibrous web of pulp fibers or an airlaid bed of pulp fibers (fluff pulp) which are guided through a calender composed of two embossing rollers. While generating an embossed pattern in the pressure area, the fibers are calendered in a punctiform or linear manner, without binding agent, and thus connected. An absorbent core has proven particularly useful that is produced by the method described in the European patent 1 032 342 B1.

Another suitable material is a polymer material with a honeycomb structure, as is described, for example, in the European patent application EP 2 444 046 A1.

The absorbent insert can be formed from an absorbent body as described above. This absorbent body can be surrounded by liquid-permeable layers in the manner of a casing, wherein the absorbent body can be fixed on one or both layers or is only loosely surrounded by these layers.

In order to prevent the absorbent insert from slipping on the blank of the sanitary bag, the absorbent insert can be fixed on the blank. The fixing can be carried out at discrete points or across the whole surface, using glue or other adhesive materials. Generally, the fixing of the blank is not necessary since the friction between blank and absorbent body is sufficient.

In a further configuration of the present invention, the sanitary bag according to the invention has means for making the opening smaller and for adapting the size of the opening to the circumference of the body part. Suitable means that can be used are a drawstring, an elastic rubber band or a binding means, with which one connecting edge can be turned back to the other one.

In a preferred embodiment, one connecting edge is turned back to the other connecting edge and is fixed in this position. To fix it, the turned-back connecting edge can, for example, have glue or another adhesive means which adheres at the point where it is fixed. The adherence should preferably be releasable. In a further configuration, a protruding binding means is mounted on the connecting edge to be turned back and has a corresponding adhesive which fixes the turned-back connecting edge and maintains the size of the opening in its adjustment. Examples of suitable binding means are elastic and non-elastic velcro tape, elastic and non-elastic adhesive tape, etc. This embodiment, in which one connecting edge is turned back to the other edge, has the advantage that the upper edge that bears on the body part remains smooth and is not puckered, as a result of which greater wearing comfort is achieved.

The present invention is explained in more detail with reference to the attached figures, in which:

FIG. 1 shows a perspective view of a sanitary bag ready for use;

FIG. 2 shows a plan view of a first embodiment of a sanitary bag in a form when not yet folded;

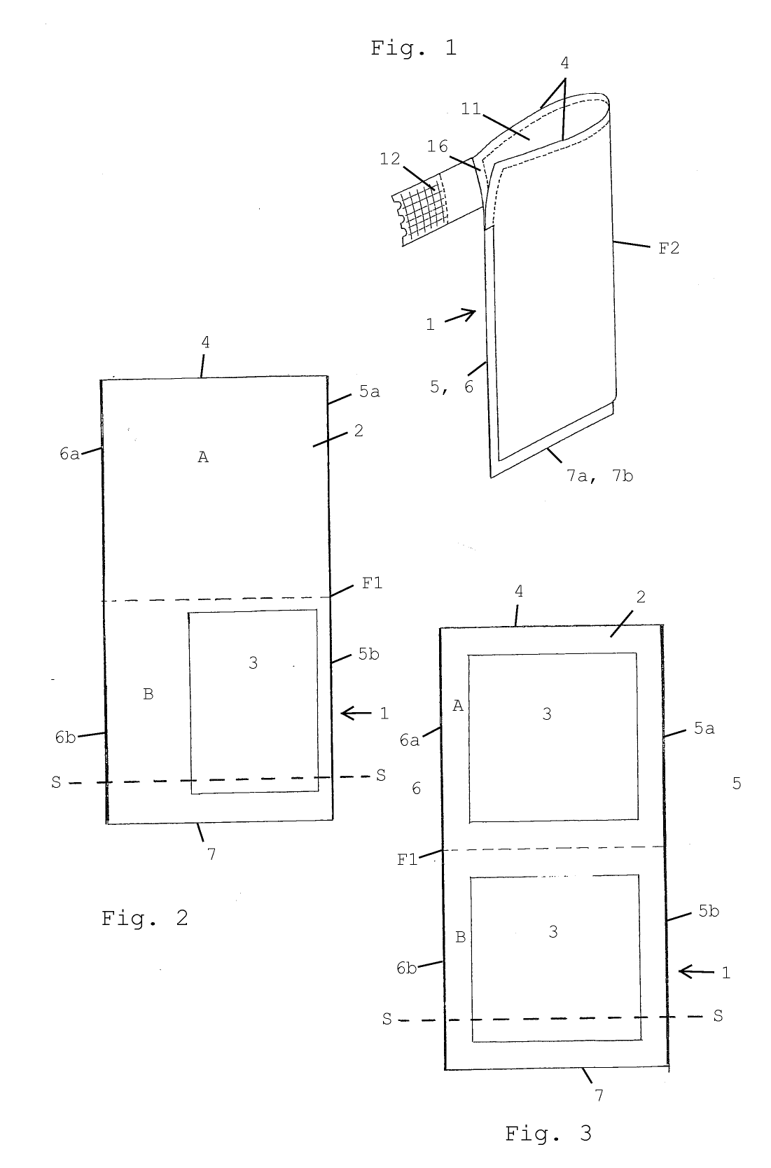

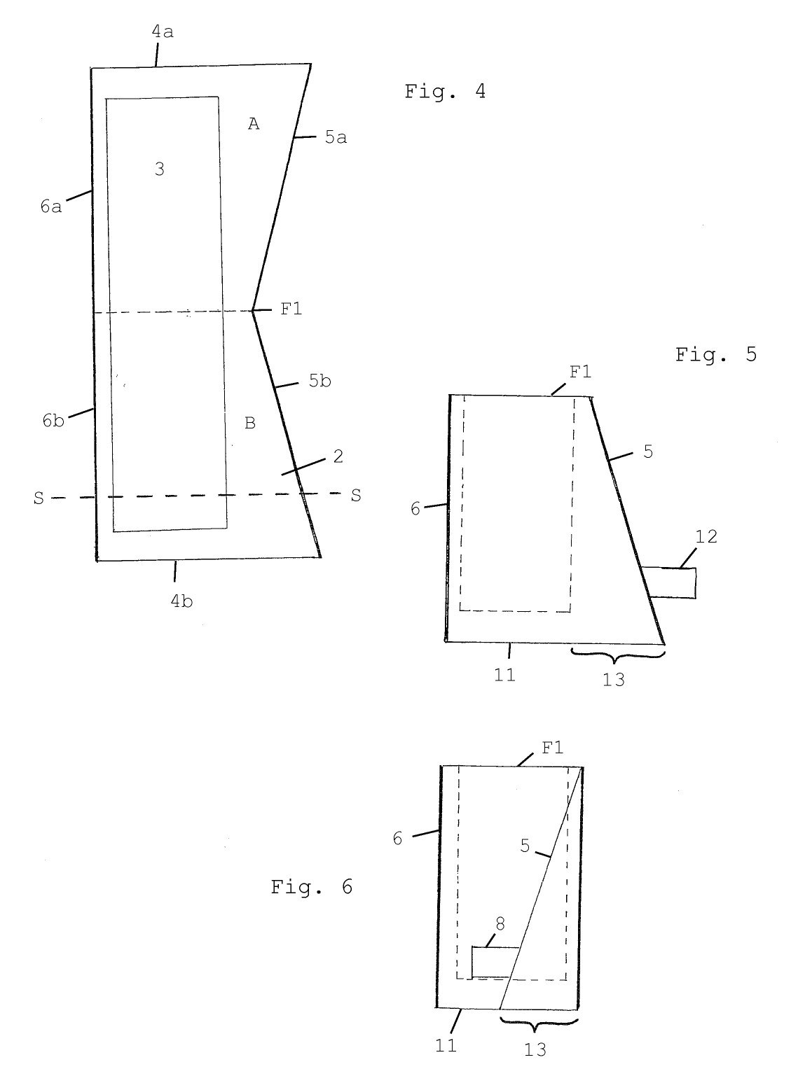

FIG. 3 shows a second embodiment of the sanitary bag in the unfolded form;

FIG. 4 shows a third embodiment of the sanitary bag in the unfolded form;

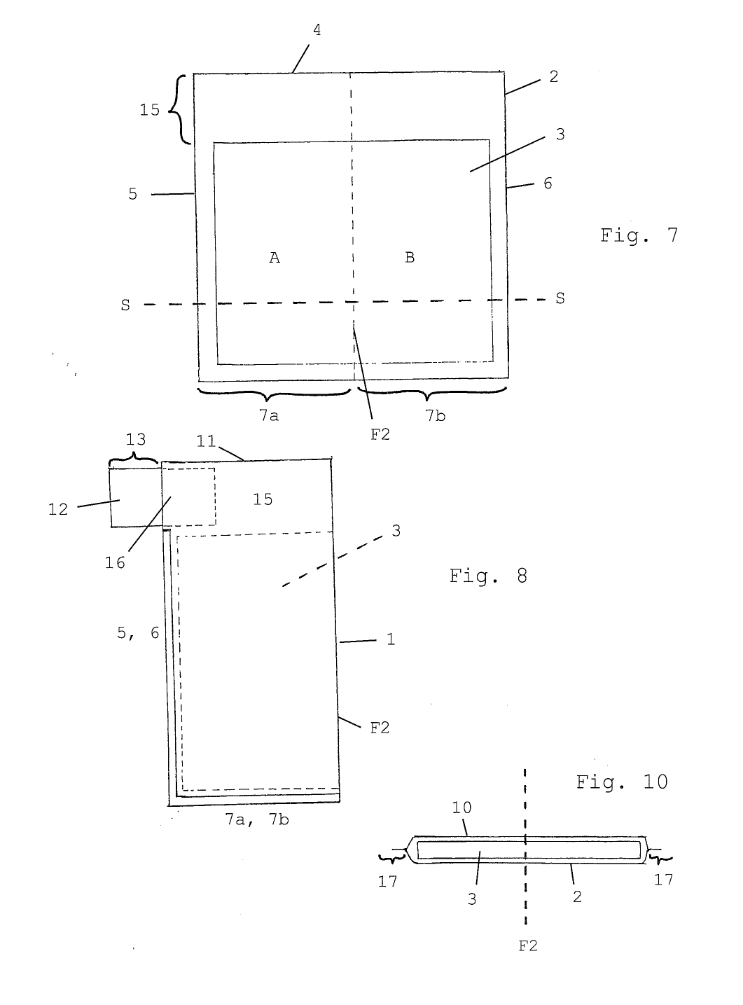

FIG. 5 shows the sanitary bag according to FIG. 4 when folded along the fold line;

FIG. 6 shows the sanitary bag according to FIG. 4 in the closed form;

FIG. 7 shows a fourth embodiment of the sanitary bag in a form when not yet folded;

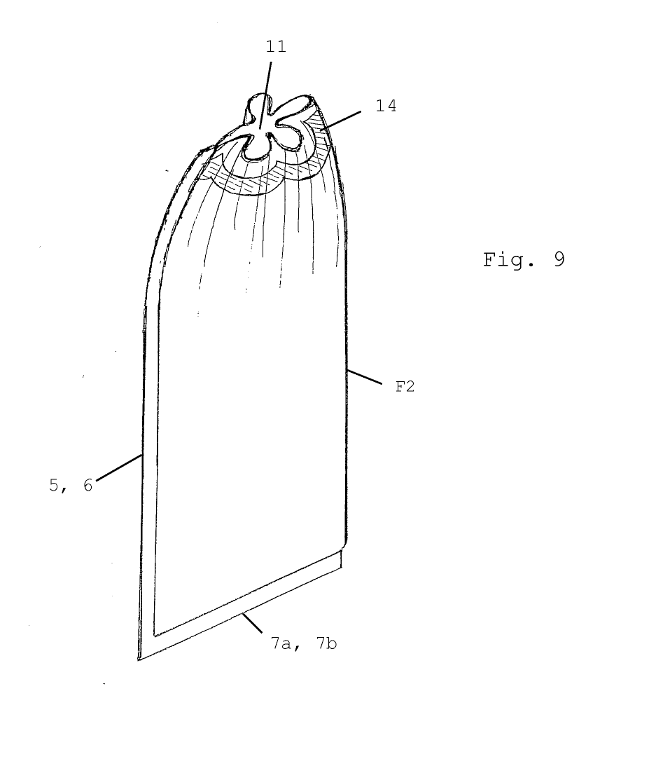

FIG. 8 shows the embodiment according to FIG. 7 in the folded form ready for use;

FIG. 9 shows a further configuration of the sanitary bag and connecting seam;

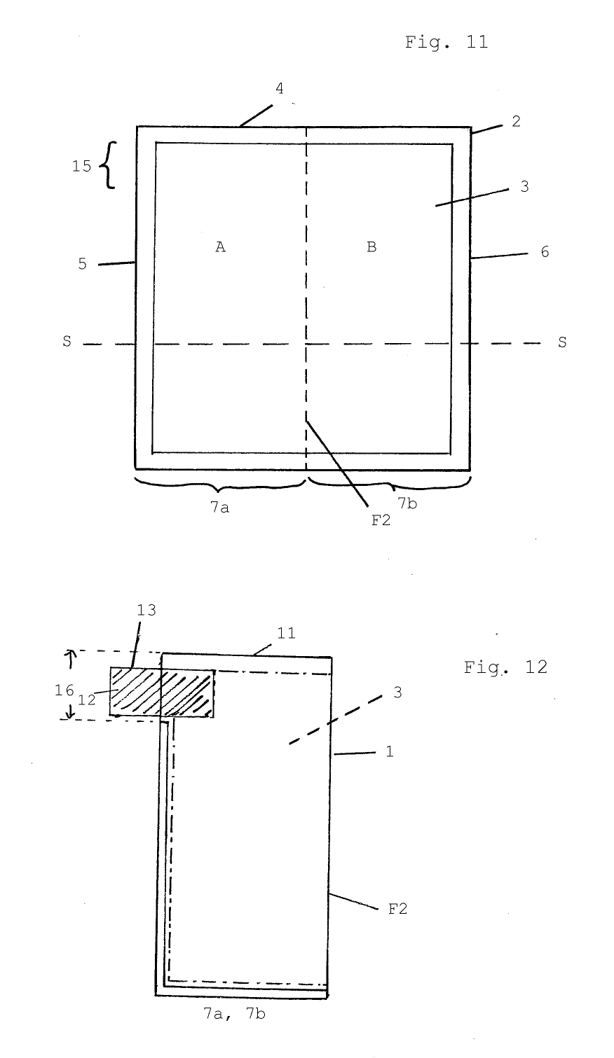

FIG. 10 shows a cross section along the line S-S in FIGS. 2, 3, 4 and 7;

FIG. 11 shows a fifth embodiment of the sanitary bag in a form when not yet folded;

FIG. 12 shows the embodiment according to FIG. 11 in the folded form ready for use.

FIG. 2 shows a possible embodiment of a sanitary bag 1 in a state when not yet folded and not yet ready for use. A generally quadrilateral blank 2 is divided along a subsequent fold line F1 into two rectangular portions A and B symmetrical with respect to the fold line F1. In the embodiment shown here, an absorbent insert 3 is arranged only on the portion B on the subsequent inside. This absorbent insert 3 extends from the fold line F1, or from a point a short distance from the fold line, in the direction of the subsequent opening edge 4 of the sanitary bag.

The blank 2 is first of all turned back along the fold line F1. The side edges 5a, 5b and 6a, 6b then lying respectively on each other are then connected to each other in a manner known per se, for example by gluing, welding or sewing.

By means of the folding and closing, a pouch or a bag is formed which is open only along one edge, i.e. has an opening 11. The opening edges are the edges 4 and 7. The fold line F1 forms the bottom of the pouch and is arranged facing away from the opening 11.

FIG. 3 shows a second possible configuration of the sanitary bag 1 according to the invention. The blank 2 itself has the same shape as in FIG. 2, but a flat absorbent body 3 is in each case located on the subsequent inside of both portions A and B of the blank 2. The absorbent bodies 3 are here arranged centrally with respect to the side edges 5a, 6a and 5b, 6b, although they can also be arranged eccentrically.

FIG. 4 shows a third configuration in which the blank 2 is formed by two portions A and B, each of them in the shape of a trapezoid. The shorter of the two parallel trapezoid edges abut each other at the fold line F1. As in all the other embodiments too, the blank can be made from a web material that has been unrolled from a reel of material.

In the third embodiment shown in FIG. 4, a single absorbent insert 3 extends transversely across the fold line F1, on the inside of the blank 2, and across large areas of both portions A and B. The blank 2 is once again turned back along the fold line F1. The abutting connecting edges 5a, 5b and 6a, 6b, respectively, are connected to each other in a manner known per se. The blank 2 is turned back such that the absorbent insert 3 preferably configured as a long rectangle is located on the inside of the formed sanitary bag. The opening 11 is formed by the two opening edges 4a, 4b.

FIG. 5 shows the sanitary bag according to FIG. 4 in its final form ready for use. The blank 2 is turned back along the fold line F1 and the connecting edges 5a, 5b, 6a, 6b lie over each other and are connected to each other in a manner known per se, resulting in a trapezoidal pouch shape with the opening 11. The flat absorbent insert 3 preferably configured as a long rectangle is arranged on the inside. It extends over both portions A and B and therefore also over the bottom of the pouch coinciding with the fold line F1. However, the absorbent insert 3 is of such length that it is at a distance from each of the two edges 4a and 4b that delimit the opening.

By means of the trapezoidal shape of the sanitary bag widening toward the opening 11, a flap 13 in the shape of a triangle is formed. For use, as is shown in FIG. 6, this flap 13 can be turned back in order thereby to adapt the opening 11 to the size of the body part. For this purpose, the connecting edge 5 running obliquely with respect to the opening 11 is turned back toward the other connecting edge 6 and is then secured in this position by means of a fixing binding element 12 on the outside of the sanitary bag.

For use, the sanitary bag 1, in the state shown in FIG. 5, is applied to the body part via the opening 11. The triangular flap 13 is then folded in the direction of the other connecting edge 6, such that the cavity formed by the blank 2 firmly encloses the body part, e.g. a man's penis. Thereafter, the binding element 12 is fixed at a suitable place on the outside of the blank. Bodily fluid, e.g. urine, can emerge from the body part and in so doing passes into the sanitary bag and is absorbed by the absorbent insert 3. The fixing by means of the binding element 12 is preferably releasable again, in order to easily remove the sanitary bag again.

However, the sanitary bag can also be held on the body part without special binding elements or fixing means. In order to adapt the opening 11 to the circumference of the body part and, if appropriate, to give the user the feeling that the sanitary bag is not going to slip, elastic materials can be applied on the outside or inside of the blank 2, along the edges 4a and 4b forming the opening 11, or at a short distance from these edges, and they keep the opening 11 small by means of suitable pretensioning.

According to FIG. 9, it is also possible for an elastic band, slightly pretensioned in the circumferential direction, to be placed along the edges 4a, 4b, i.e. around the opening 11, and through this edge portion of the blank, so as to make the size of the opening 11 variable.

In the embodiment shown in FIG. 7 and FIG. 8, the absorbent insert 3 extends in one piece across both portions A and B and across the fold line F2, which runs transversely with respect to the opening of the blank 2. Here too, the blank 2 is folded along the fold line F2, which forms a right angle with the edge 4 for the opening. The two lateral edges 5 and 6 and the two edges 7a and 7b forming the bottom of the bag are connected to each other respectively. In the embodiment shown here, the absorbent insert 3 extends over almost the entire height of the blank 2, and in particular it extends to a point close to the bottom formed by the edges 7a, 7b. However, the absorbent insert 3 is spaced apart from the vertical edges 5 and 6 and also from the bottom edges 7a, 7b, such that, when these edges 5, 6, 7a, 7b or edge areas are connected, the absorbent insert 3 is not included, i.e. not bound in with them. The edge area as far as the opening edge 4 forms a bearing surface 15 of the sanitary bag on the body part. The size of the opening 11 is also fixed by the length of this bearing surface 15.

The connecting edges 5, 6 and 7a, 7b in the embodiment according to FIGS. 1, 7 and 8, and the connecting edges 5a, 5b, 6a, 6b in the embodiments according to FIGS. 2 to 6, are connected to each other by customary methods known from the prior art. These connecting edges should preferably be leaktight to liquids, so as to prevent bodily fluid from escaping via these edges. Such a connection can be obtained, for example, by the edges of the blanks 2, 10 being welded to each other. Welding is possible, for example, when the blanks are made of thermoplastic material, with the superposed edges being melted by application of heat and joining together by fusing.

In the particular configuration shown in FIG. 1 and FIG. 8, the connecting edge extends along the bottom and along the vertical connecting edges 5, 6, which run parallel to the fold edge F2. In the embodiment shown in FIG. 1 and FIG. 8, a connection of the two vertical edges 5, 6, starting from the bottom connecting edge 7a, 7b, does not take place over the entire length as far as the opening edge 4. Instead, the connection ends at a distance before the opening edge 4, such that an edge portion 16, which extends from the weld seam to the opening 11, remains unclosed. This edge portion 16, unclosed toward the side, acts as a widening of the opening 11, thereby simplifying the handling of the sanitary bag.

Likewise in FIG. 1 and FIG. 8, at the height of the edge portion 16, a binding element 12 can be applied either in the area of the edge 5 or of the edge 6, which binding element 12 is fastened to the surface 15 lying opposite the edge in order to close and adapt the size of the opening 11. The binding element 12 is preferably releasable again, for example a Velcro fastener or an adhesive fastener. The binding element 12 can be elastic in its longitudinal direction, as a result of which the size of the opening 11 adapts even better to the body part during use.

FIG. 9 shows an embodiment of the sanitary bag in which the vertical edges 5, 6 are connected, that is to say closed, all the way to the opening 11. The size of the opening can be adapted to the body part by an elastic material applied to the inside or outside of the sanitary bag, close to or directly on the edge 4. In one possible configuration, an elastic drawstring 14 is drawn in near the edge 4.

In the simplest configuration, the bag-shaped sanitary bag has a blank 2 in which an absorbent body 3 is arranged. The absorbent body takes up the discharged bodily fluid and stores it. The blank 2 is intended to prevent the bodily fluid from escaping to the outside, i.e. in the direction of the clothes, and thus soiling the clothes.

Likewise, in all the embodiments, the sanitary bags according to the invention can also be multilayered. In one possible configuration, a further blank 10 is arranged on the inside over the absorbent body 3. This further blank 10 therefore forms the inside or inner wall of the sanitary bag during use. In this connection, FIG. 10 shows a cross section along the line S-S and depicts such a three-layered structure of the sanitary bag. The absorbent core 3 is formed by the blank 2, which forms the outwardly facing surface of the sanitary bag, and by the further blank 10, which forms the inwardly facing surface of the sanitary bag. The edges 5a, 5b and 6a, 6b of the blank 2 and the edges 5a, 5b and 6a, 6b of the further blank are connected to each other at the connection 17 and enclose the absorbent body 3. The edges of the blank 2 and of the further blank 10 can be connected in a manner known per se. It is not necessary that this connection 17 is watertight. In a preferred embodiment, the edges are connected to each other loosely but not releasably, such that a relatively soft connecting edge 17 is formed. In the area of the opening edge 4, the edge 17 bears on the body part, and a soft structure pleasant to the skin is advantageous in particular for this bearing surface. In the cross section shown in FIG. 10, the absorbent body 3 is encased by the blanks 2 and 10 without being connected to them. It is also possible for the absorbent body 3 to be connected over all of its surface, or over part of its surface, to a blank 2, 10, or at the same time to both blanks 2, 10.

Another possible way of fixing the absorbent body is for the absorbent body 3 to be enclosed immovably by the blanks 2 and 10, with the connecting edges 5 and 6 at only a slight distance from the absorbent body.

If the multilayered structure according to FIG. 10 is folded along the fold lines F1 or F2, the connections at the edges 5, 6 and 7a, 7b or 5a, 5b and 6a, 6b lie over each other and are connected to each other in order to finish the sanitary bag. As has already been described, this connection is preferably watertight.

The embodiment shown in FIG. 11 and FIG. 12 differs from the embodiment in FIG. 7 and FIG. 8 in terms of the arrangement of the absorbent insert 3. In this embodiment too, the latter extends in one piece across both surface portions A and B and across the fold line F2 of the blank 2. The absorbent insert extends in the direction of the opening edge 4 beyond the length of the connecting seam of the edges 5, 6. In the embodiment shown here, the absorbent insert ends at the upper edge of the binding element 12.

The sanitary bag according to the invention is preferably produced from web material that is unwound from a storage reel and delivered to the machine. The material for the blank 2, for the absorbent insert 3 and also for optional further layers or blanks 10 is delivered as web material wound onto reels. The absorbent insert 3 is placed on the web material of the blank 2, the optional further layer is arranged thereover as a further blank 10 either at the same time or in a following step. The absorbent insert 3 and the web material for the blank 2 and the web material for the optional further blank 10 are cut to a suitable size in a subsequent method step. To facilitate the further production process, the superposed edges can be connected to each other, in order thereby to prevent the superposed blanks from slipping. The connection can be achieved, for example, by means of the ultrasonic welding already described above.

Thereafter, the blank produced in this way is folded along the fold line F1 or F2, and the edges 5a, 5b and 6a, 6b or 5, 6 and 7a, 7b thus coming to lie over each other are connected to each other to form a watertight connection. Preferably, the binding element 12 or the elastic material 14 is applied beforehand, i.e. before the edges are connected.

### LIST OF REFERENCE SIGNS

1 sanitary bag

2 blank, outer layer

3 absorbent insert

4 opening edge

5 connecting edge

5a, 5b connecting edge

6 connecting edge

6a, 6b connecting edge

7 edge, opening edge

7a, 7b connecting edge

10 blank, inner layer

11 opening

12 binding element

13 flap

14 elastic material

15 bearing surface

16 unclosed edge

17 connection

F1 fold edge

F2 fold edge

1. A sanitary bag for receiving at least one part of the body, in particular the penis, which sanitary bag is composed at least of a blank (2) with two portions (A, B) which each have the shape of a quadrilateral and are folded along a fold line (F1, F2), wherein the two portions A and B have the same length from the fold line (F1, F2) to their edge (4, 5, 6, 7) facing away from the fold line, characterized in that an absorbent insert (3) is arranged on the inside of the blank (2).

2. The sanitary bag as claimed in claim 1, characterized in that a further blank (10) is arranged on the inside over the absorbent insert (3).

3. The sanitary bag as claimed in claim 2, characterized in that the blanks (2 and 10) have the same size.

4. The sanitary bag as claimed in claim 1, characterized in that the blank (2) is made of a material impermeable to liquids.

5. The sanitary bag as claimed in claim 4, characterized in that the blank (2) is made of a breathable material chosen from perforated films, breathable SMS films, nonwovens of natural or synthetic fibers, laminates of nonwovens and breathable films and/or BTBS films.

6. The sanitary bag as claimed in claim 2, characterized in that the further blank (10) is made of a material permeable to liquids.

7. The sanitary bag as claimed in claim 6, characterized in that the material permeable to liquids is an elastic nonwoven fabric.

8. The sanitary bag as claimed in claim 1, characterized in that a single absorbent insert (3) extends over both portions A and B, and its edge is offset inward with respect to the edges (4, 5, 6) of the blank (2).

9. The sanitary bag as claimed in claim 2, characterized in that the blanks (2 and 10) are connected to each other exclusively in edge areas.

10. The sanitary bag as claimed in claim 1, characterized in that the absorbent insert (3) is made of web material from oriented and/or unoriented fibers, in particular of nonwovens from polymers and/or pulp and pulp-related materials.

11. The sanitary bag as claimed in claim 10, characterized in that the absorbent insert (3) is produced from a fibrous web of cellulose fibers or an airlaid bed of cellulose fibers which are guided through a calender composed of two embossing rollers.

12. The sanitary bag as claimed in claim 2, characterized in that connecting edges (5a, 5b and 6a, 6b) superposed by folding the blanks (2, 10) along the fold line (F1) are connected to each other in a watertight manner.

13. The sanitary bag as claimed in claim 2, characterized in that the connecting edges (5, 6 and 7a, 7b) superposed after folding the blanks (2, 10) along the fold line (F2) are connected to each other in a watertight manner.

14. The sanitary bag as claimed in claim 13, characterized in that the connecting edge (5, 6) is spaced apart from the opening edge (4) of the opening.

15. The sanitary bag as claimed in claim 14, characterized in that its opening is formed by an opening edge (4) and by a portion (16) adjoining the latter.

16. The sanitary bag as claimed in claim 1, characterized in that a binding element (12) is arranged on the upper portion of the connecting edge (5, 6).

17. The sanitary bag as claimed in claim 1, characterized in that the one closure edge (6) is turned back to the other closure edge (5) and is fixed in this position.

18. Use of the sanitary bag as claimed in claim 1 as an incontinence product.
